(19) United States
(12) Patent Application Publication
(10) Pub. No.: US 2010/0204717 A1
(43) Pub. Date: Aug. 12, 2010

(54) Surgical Device for Multiple Clip Application

(75) Inventor: Bryan D. Knodel, Flagstaff, AZ (US)

Correspondence Address: CARDICA, INC., 900 SAGINAW DRIVE, REDWOOD CITY, CA, 94063, US

(73) Assignee: Cardica, Inc., Redwood City, CA (US)

(21) Appl. No.: 12/370,576

(22) Filed: Feb. 12, 2009

(51) Int. Cl. A61B 17/10

(52) U.S. Cl. 606/143; 606/142

(57) ABSTRACT

An example of a surgical clip applier may include a housing with a passage defined therein, the passage having a longitudinal centerline; a plurality of clips held within the passage, each clip lying in a plane angled relative to the longitudinal centerline of the passage; a pusher slot defined in the housing; and a pusher slidable along the pusher slot, wherein distal motion of the pusher urges the distalmost clip in the passage out of the passage in a direction angled relative to the longitudinal centerline of the passage. Another example of a surgical clip applier may include a first jaw having a control slot defined therein; a second jaw having a control slot defined therein; and a control arm extending into the control slot of each jaw; wherein the control arm is movable along the control slots in a first direction to close the jaws and is movable along the control slots in a second direction to open the jaws.

### FIELD OF THE INVENTION

The present invention relates generally to a surgical clip applier and a method for surgical clip application.

### BACKGROUND

Traditionally, suture has been utilized to close wounds and incisions, to attach separate tissue structures to one another, and to perform other medical and surgical functions. However, suturing requires skill to perform, particularly suturing procedures that are complex, time-consuming and/or difficult. Further, suturing may be impractical or unfeasible in certain situations. For example, in a minimally-invasive surgical procedure through a small opening (often referred to as an access port) in the patient's body, that port may not be large enough to allow suturing to be performed through it.

Surgical staplers and clip appliers have been utilized to overcome some of the difficulties associated with suturing. However, a large subset of surgical staplers and clip appliers known in the art are single-use devices, capable of deploying only a single clip or set of clips at one time. To deploy another clip or set of clips, a new device must be utilized. Where multiple clips must be placed at different locations in the body, such use of multiple tools is time consuming, expensive and wasteful. Some surgical staplers and clip appliers known in the art are capable of deploying multiple clips, one after the other, in different locations in the body. However, such tools are complex and bulky, and may be incapable of deploying clips small enough to be useful in some minimally invasive surgical procedures.

### BRIEF DESCRIPTION OF THE DRAWINGS

FIG. 1 is a perspective view of an exemplary surgical clip applier.

FIG. 2 is a cutaway perspective view of the distal end of an exemplary effector of the surgical clip applier of FIG. 1.

FIG. 2A is a side cutaway view of the exemplary effector of the surgical clip applier of FIG. 1.

FIG. 3 is a perspective view of a portion of the distal end of the exemplary effector of the surgical stapler of FIG. 1, with a clip in ready position.

FIG. 4 is a top view of the distal end of the exemplary effector of the surgical stapler of FIG. 1, with the jaws open.

FIG. 5 is a perspective view of a portion of the distal end of the exemplary effector of the surgical stapler of FIG. 1, with a closed clip between the jaws.

FIG. 6 is a top view of a portion of the distal end of the exemplary effector of the surgical stapler of FIG. 1, with a closed clip between the jaws.

FIG. 7 is a perspective view of the exemplary effector of the surgical clip applier of FIG. 1.

The use of the same reference symbols in different figures indicates similar or identical items.

### DETAILED DESCRIPTION

Clip Applier

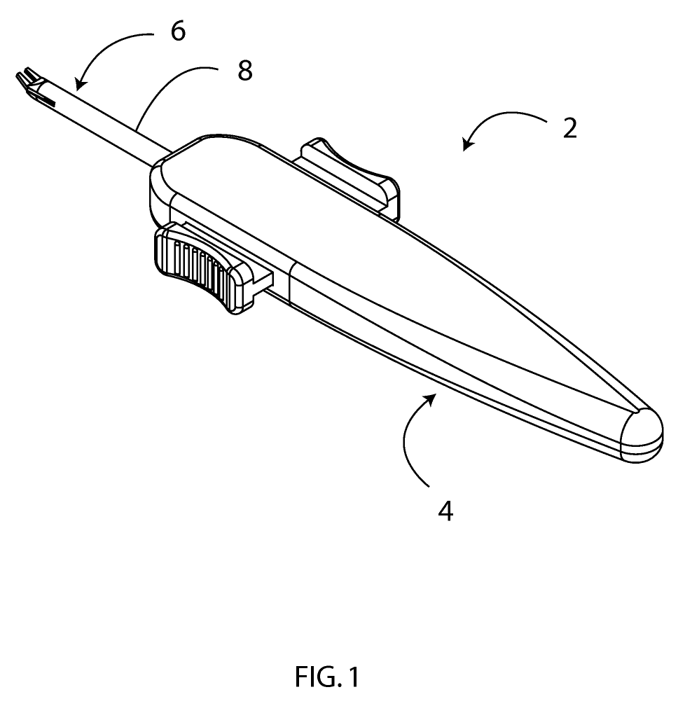

Referring to FIG. 1, a surgical clip applier 2 includes a handle 4 connected to an effector 6. The handle 4 may be connected directly to the effector 6, or may be connected to a shaft 8 that in turn is connected to the effector 6. The shaft 8 may be rigid, articulated or flexible. The handle 4 may have any suitable configuration, as described in greater detail below. The effector 6 may be sized to pass through an access port in a patient for use in a minimally-invasive surgical procedure. The effector 6 may be sized and shaped to allow it to be inserted through an access port in a patient of 5 mm in diameter or less. Alternately, the effector 6 may be sized and/or shaped differently. The effector 6 may be substantially rigid, substantially flexible, or a combination of both. The handle 4 may include one or more triggers, levers, knobs, buttons or other input features used to actuate and/or control the effector 6.

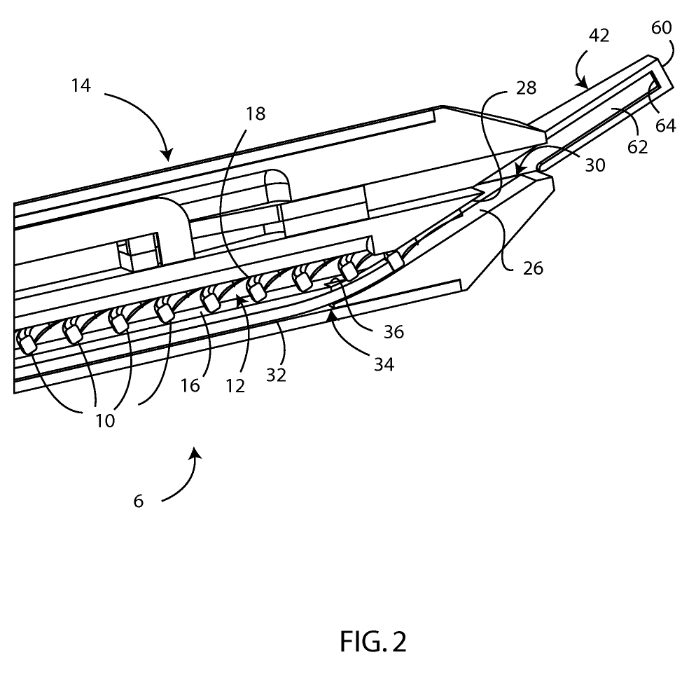

Referring to FIG. 2, the effector 6 may hold one or more clips 10 within a passage 12 in a housing 14. The housing 14 may be elongated longitudinally, and may have a longitudinal centerline. The passage 12 in the housing 14 may itself have a longitudinal centerline that is offset from and generally parallel to the longitudinal centerline of the housing 14. Alternately, the passage 12 may be located in and/or oriented within the housing 14 in any other suitable manner. The passage 12 may have a lower surface 16 and an upper surface 18. One or more clips 10 are located within the passage 12. The clips 10 may be oriented at an angle to the longitudinal centerline of the passage 12 and/or the longitudinal centerline of the housing 14. The clips 10 may be constrained within the passage 12 by the surfaces 16, 18, as well as the lateral surfaces of the passage 12. The clips 10 may be independent from one another, and placed adjacent to one another. Alternately, the clips 10 may be connected to or associated with one another in any suitable manner. As one example, the clips 10 may be coated with parylene such that they are held together as a single assembly, in order to facilitate manufacture of the clip applier 2. As another example, one or more clips 10 may be frangibly connected to a wire or backbone (not shown) from which each clip 10 may be sheared upon deployment, in order to facilitate manufacture of the clip applier 2.

Referring also to FIG. 2A, a clip pusher 20 may be located in the passage 12, proximal to the clip or clips 10. The clip pusher 20 may contact the most proximal clip 10 in the passage 12, and exert a force on that most proximal clip 10 in the distal direction. The distal force may be transmitted from each clip 10 to the clip 10 distally next in line, such that the clip pusher 20 exerts a distal force on all of the clips 10. The clip pusher 20 may be biased distally, or may be selectively urged distally. Where the clip pusher 20 is biased distally, such bias may be provided by a compression spring 22, by a pneumatic force, by a magnetic or electromagnetic actuator, a solenoid, or any other suitable structure or mechanism. The distal end of the compression spring 22 may abut or may be fastened to the clip pusher 20, and the proximal end of the compression spring 22 may abut or be fixed to a wall 24 or other portion of the housing 14 such that the proximal end of the compression spring 22 is held in a substantially fixed location. However, the compression spring 22 may be arranged differently relative to the housing 14, if desired. Where the clip pusher 20 is selectively urged distally, the handle 4 may be used to control the timing of the application of distal force to the clip pusher 20, which may be applied by a compression spring 22, by a pneumatic force, by a magnetic or electromagnetic actuator, a solenoid, or any other suitable structure or mechanism.

A lower ramp 26 may be located at the distal end of the passage 12. The lower ramp 26 advantageously is oriented at the same angle to the longitudinal centerline of the housing 16 as the clip or clips 10. The distalmost clip 10 in the passage may be pressed against the ramp 26 by the compressive force applied to that clip 10 by the clip pusher 20. A upper ramp 28 may be generally parallel to the lower ramp 26, and spaced apart from the lower ramp 26 by a distance slightly greater than the thickness of the clip or clips 10. Friction between the distalmost clip 10 and the ramps 26, 28 holds the distalmost clip 10 in place. Optionally, a detent or other mechanism may be used to hold the distalmost clip in place. An opening 30 is located between the distal ends of the ramps 26, 28. Clips 10 are sequentially urged through that opening 30, as described in greater detail below.

A pusher 32 is slidable along a pusher slot 34 in the housing 14. The pusher 32 may be generally elongated, and may have a generally rectangular cross-section. Alternately, the pusher 32 may be shaped differently. The pusher slot 34 may be generally parallel to and underneath the passage 12. Alternately, the pusher slot 34 may be located and/or oriented differently. The distal end of the pusher slot 34 includes an aperture 36 connecting the pusher slot 34 to the passage 12. The aperture 36 is preferably oriented such that the distal end of the pusher 32 extends out of the aperture 36 onto the lower ramp 26. Alternately, the pusher slot 34 is omitted, and the pusher 32 is slidable along a trench or other feature in the housing 14.

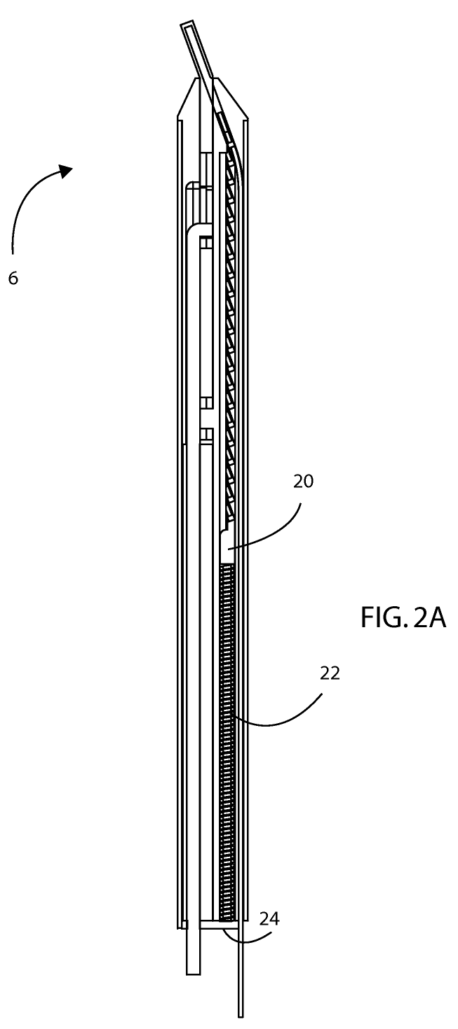

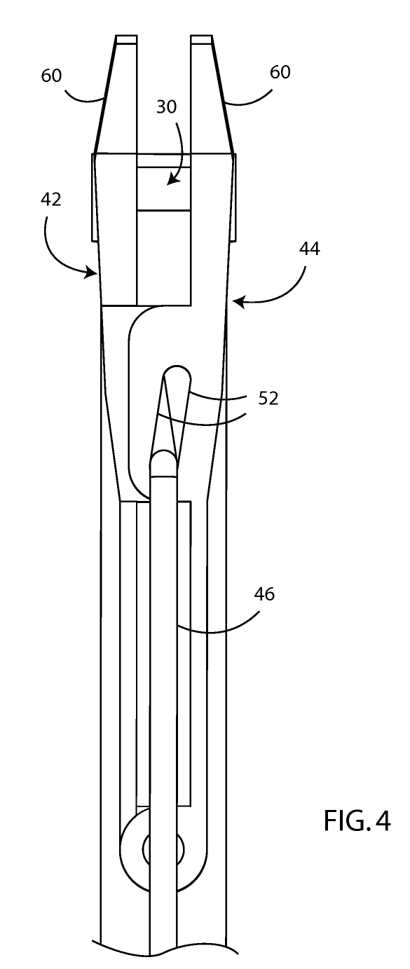

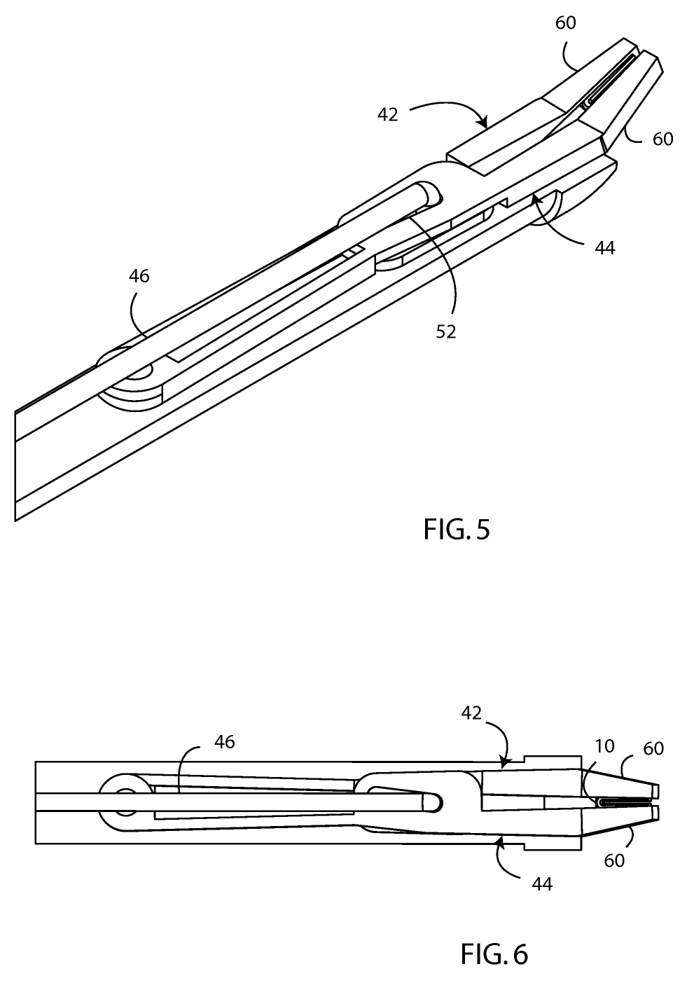

Referring also to FIGS. 3-6, the effector 6 includes an actively-controlled crimper 40. The crimper 40 includes a first jaw 42, a second jaw 44, and a control arm 46. Each jaw 42, 44 may be substantially planar, and the jaws 42, 44 may be positioned such that the second jaw 44 rests on the upper surface of the first jaw 42. Alternately, at least one jaw 42, 44 may be shaped or oriented differently. The proximal end of each jaw 42, 44 may be configured to pivot about a post 48 within the housing 14, where the post 48 has a substantially fixed location within the housing 14. For example, the proximal end of each jaw 42, 44 may include a generally circular opening 50 that receives the post 48 therein. Moving distally to a middle portion of each jaw 42, 44, an angled control slot 52 is defined in each jaw 42, 44. The control slot 52 of each jaw 42, 44 is oriented such that the distal end of the control slot 52 is located laterally outward from the proximal end of the control slot 52. The control slot 52 of the first jaw 42 may be oriented laterally in the opposite direction as the control slot 52 of the second jaw 44. Moving distally, each jaw 42, 44 ends in a finger 60, where the fingers 60 are configured to receive a clip 10 therebetween. Each finger 60 may be angled relative to the longitudinal centerline of the effector 6 approximately the same amount as the lower ramp 26, and may extend outward from the opening 30 such that a clip 10 can slide smoothly along the lower ramp 26, out of the opening 30, and into the space between the fingers 60. The inner surface of each finger 60 may include a trench 62 defined therein. Each trench 62 may be sized to be substantially as wide as a clip 10. Each trench 62 may be deep enough such that the distal end 64 of each trench 62 acts as a stop, such that a clip 10 is advanced between the fingers 60 until the distal end of the clip 10 encounters the ends 64 of the trenches 62 in the fingers 60.

The arm 46 may extend generally parallel to the jaws 42 along at least part of its length. The distal tip 66 of the arm 46 is bent downward or otherwise configured to enter both control slots 52 of the jaws 42, 44. Initially, the distal tip 66 of the arm 46 is located at the proximal end of each slot 52, and the fingers 60 of the jaws 42, 44 are spaced apart from one another. Alternately, a feature 66 is located on the control arm 46 other than at its distal end, and extends downward into both control slots 52 of the jaws 42, 44.

The clips 10 may be generally U-shaped, or otherwise configured. Each clip 10 may lie substantially in a single plane. That is, each clip 10 is shaped such that a single plane extends through and substantially bisects the entire clip 10. Alternately, at least one clip 10 does not lie substantially in a single plane. The longitudinal and lateral dimensions of the clips 10 overall may both be substantially larger than the height of the clips 10. Alternately, the clips 10 may be sized differently. Advantageously, the clips 10 may be plastically deformable. That is, the clips 10 may undergo a permanent deformation when subjected to a stress exceeding its yield value. In other words, plastic deformation is deformation that remains after the load that caused it is removed, or that would remain if the load were removed. If so, the clips 10 may be fabricated from stainless steel, titanium or any other suitable plastically-deformable material. Alternately, the clips 10 may be elastically deformable. If so, the clips 10 may be fabricated from nickel-titanium alloy or any other suitable elastic or superelastic material. Each clip 10 may be fabricated from a single wire or other piece of material, having a rectangular, circular or other cross-section. However, the clips 10 may be fabricated in any suitable manner. The cross-section of each clip 10 may be substantially constant along the entire clip 10, or may vary at different locations along the clip 10. For example, the cross-sectional area of the clip 10 at certain locations may be less than at other locations, in order to promote bending in those locations having a lesser cross-sectional area. The cross-sectional shape of the clip 10 may be square, rectangular, circular, oval or any other suitable shape, and may be substantially constant along the entire clip 10 or vary at different locations along the clip 10.

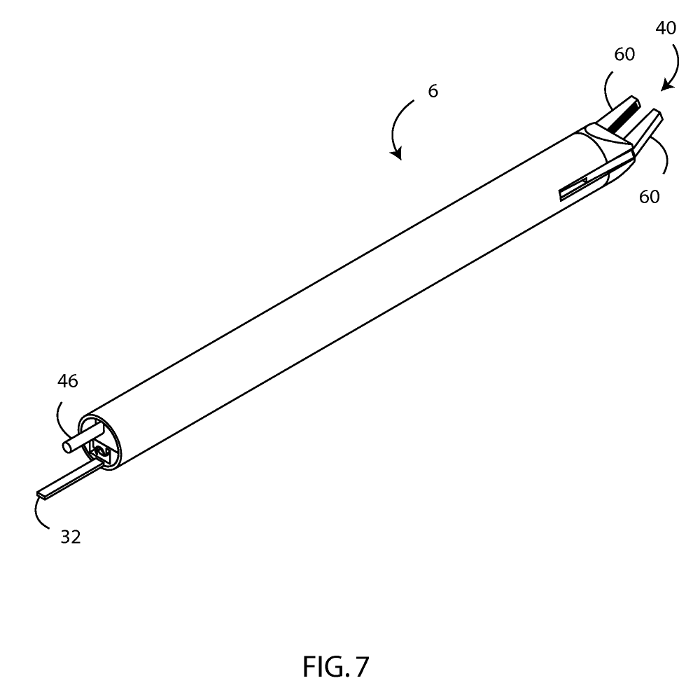

The handle 4 may include any mechanism, mechanisms, structure or structures configured to actuate the effector 6. Referring to FIG. 7, the proximal end of the pusher 32 and the proximal end of the control arm 46 may extend proximally out of the effector 6. The handle 4 may be configured in any suitable manner to control the motion of the pusher 32 and control arm 46. As one example, the pusher 32 and/or control arm 46, or structures or mechanisms connected to them, may extend through the shaft 8 to the handle 4. The handle 4 may include any suitable mechanism or mechanisms that provide for control of the pusher 32 and control arm 46, and may include a source of stored energy for actuating the effector 6. The source of stored energy may be mechanical (such as a spring), electrical (such as a battery), pneumatic (such as a cylinder of pressurized gas) or any other suitable source of stored energy. The source of stored energy, its regulation, and its use in actuating an effector 6 may be as described in U.S. patent application Ser. No. 10/392,336, filed on Mar. 19, 2003, or U.S. patent application Ser. No. 11/054,265, filed on Feb. 9, 2005, which are herein incorporated by reference in their entirety. The handle 4 may instead, or also, include a connector or connectors suitable for receiving stored energy from an external source, such as a hose connected to a hospital utility source of pressurized gas or of vacuum, or an electrical cord connectable to a power source.

Alternately, the handle 4 may be omitted, and the effector 6 may be actuated directly by a surgical robot such as the DaVinci® surgical robot of Intuitive Surgical, Inc. of Sunnyvale, Calif. The shaft 8 may be utilized, if desired, or the effector 6 may be mounted directly on an arm of the surgical robot. The surgical robot may provide all energy needed to actuate the effector 6, and may directly control the actuation of the effector 6.

Optionally, the effector 6 may include a cutaway, trough, lumen, ring or other feature (not shown) to allow the effector 6 to follow a guidewire to a treatment site.

Operation

The operation of the clip applier 2 is described with regard to a generic surgical procedure. The clip applier 2 may be used in the course of any suitable surgical procedure, whether that surgical procedure is minimally-invasive or open, and whether the clip applier 2 is configured for manual or robotic actuation. For example, the surgical stapler 2 may be used to staple wounds or incisions in the skin together, for cardiac surgery, for hernia repair, for abdominal wall closure, for anti-reflux or other bariatric procedures, for intestinal repair, for dura mater surgery or other brain surgery, for aneurysm closure, for anastomosis, or for any other suitable medical use.

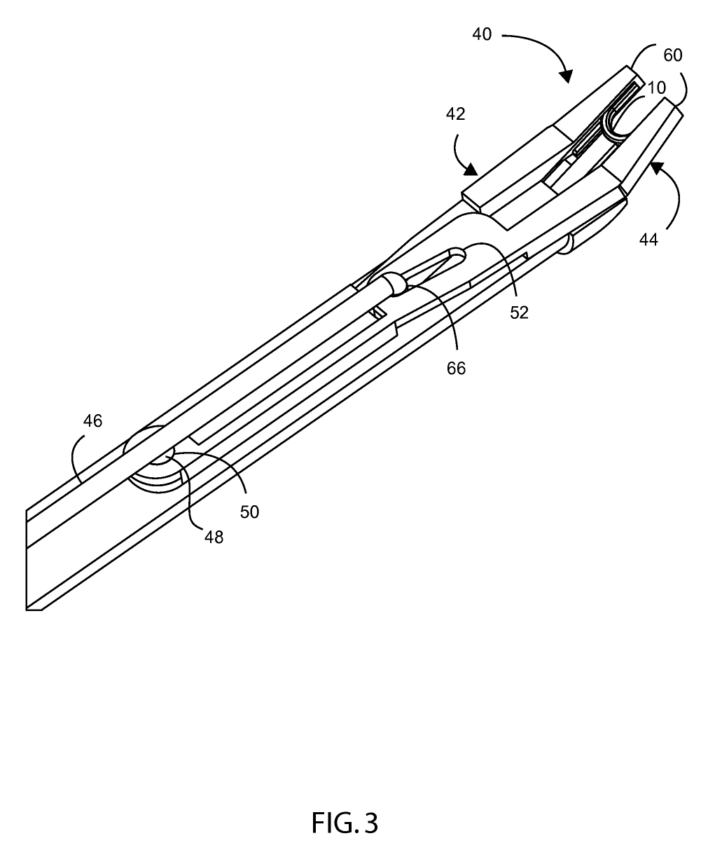

The distal end of the effector 6 may be placed in proximity to the tissue to be clipped. For example, the fingers 60 may be placed around a blood vessel. For clarity in describing the operation of the stapler 2, that tissue is not shown in the figures. Referring to FIGS. 2 and 4, the effector 6 is in an initial state. In the initial state, the fingers 60 are spaced apart from one another in an open configuration, prepared to receive a clip 10. The user then actuates the handle 4 and/or other component of the clip applier 2 to begin the deployment sequence. As described above, the handle 4 or other mechanism controls the motion of the components of the effector 6 in any suitable manner. First, the pusher 32 is advanced distally. As the pusher 32 advances, it contacts the proximal surface of the distalmost clip 10, then pushes that clip along the lower ramp 26 and through the opening 30. As the clip 10 moves through the opening 30, the lateral surfaces of the clip 10 enter the trenches 62 of the fingers 60, such that the fingers 60 can effectively hold the clip 10 therebetween. Motion of the clip 10 ceases when the pusher 32 ceases its distal motion, or when the distal end of the clip 10 encounters the distal end 64 of at least one trench 62. The clip 10 is thereby in position for placement on tissue. This position of the clip 10 may be referred to as the “ready position.” The clip 10 may be placed around a blood vessel, onto tissue, or in any other suitable position before or after the clip 10 has reached the ready position.

The control arm 46 is then actuated to move distally; advantageously, the control arm 46 also moves substantially linearly. As the control arm 46 moves distally, the distal tip 66 of the control arm 46 moves distally as well. The distal tip 66 is located within the control slots 52 of the jaws 42, 44. As that distal tip 66 moves distally along a generally straight line, it encounters the inner surface of each angled control slot 52, thereby causing each jaw 42, 44 to move inward such as by rotating about the post 48. Thus, as the distal tip 66 moves distally, the fingers 60 move closer together, closing the clip 10. The distal tip 66 may travel all the way to the distal end of at least one control slot 52, or may be controlled by the handle 4 to move a shorter distance along at least one control slot 52. The distal tip 66 moves distally until the clip 10 is completely closed. After that deployment, the distal tip 66 is moved proximally, causing the fingers 60 to move apart and release the clip 10. The control arm 46 is moved proximally until the jaws 42, 44 return to their initial, open position in which the fingers 60 can receive another clip 10 between them. The jaws 42, 44 may be actively controlled at all times. “Active control” means that the opening and closing of the jaws 42 is controlled solely by the control arm 46, and is not dependent on or responsive to other input, such as contact between one or more of the fingers 60 and tissue. Alternately, the jaws 42, 44 may be otherwise controlled.

The pusher 32 is also moved proximally to its initial position. This motion may occur at any time after the clip 10 has been pushed into its ready position between the fingers 60. For example, the pusher 32 may move proximally before, during or after the distal tip 66 of the control arm 46 moves distally to close the clip 10 between the fingers 60. The clip pusher 20 may apply a compressive force collectively to all of the clips 10 in the passage before, during and after each distalmost clip 10 is urged into the ready position. As a result, as the pusher 32 moves proximally, the distalmost remaining clip 10 in the passage 12 is urged distally against the pusher 32. When the distal tip of the pusher 32 moves beneath the distalmost remaining clip 10, all of the clips 10 advance in the passage 12, such that the distalmost remaining clip 10 is pushed against the lower ramp 26. Thus, the clips 10 may be advanced without the need for a belt, carrier or other mechanism to engage them each individually and separately and move them distally. The pusher 32 may then cease its proximal motion. At the time the pusher 32 ceases to move proximally, the distal end of the pusher 32 may be located in the passage 12, or may be located in the pusher slot 34. At this time, the effector 6 is ready for another actuation at any other suitable location in the patient.

The terms “upper,” “lower,” “upward,” “downward,” “up,” “down,” “below,” “above” and the like are used solely for convenience in this document; such terms refer to directions on the printed page and do not limit the orientation of the clip applier 2 as a whole, or of the handle 4, effector 6 and/or shaft 8, in use. While the invention has been described in detail, it will be apparent to one skilled in the art that various changes and modifications can be made and equivalents employed, without departing from the present invention. It is to be understood that the invention is not limited to the details of construction, the arrangements of components and/or the details of operation set forth in the above description or illustrated in the drawings. Headings and subheadings are for the convenience of the reader only. They should not and cannot be construed to have any substantive significance, meaning or interpretation, and should not and cannot be deemed to be limiting in any way, or indicate that all of the information relating to any particular topic is to be found under or limited to any particular heading or subheading. The contents of each section of this document are merely exemplary and do not limit the scope of the invention or the interpretation of the claims. Therefore, the invention is not to be restricted or limited except in accordance with the following claims and their legal equivalents.

1. A surgical clip applier, comprising:
a housing with a passage defined therein, said passage having a longitudinal centerline;
a plurality of clips held within said passage, each said clip lying in a plane angled relative to said longitudinal centerline of said passage;
a pusher slot defined in said housing; and
a pusher slidable along said pusher slot, wherein distal motion of said pusher urges the distalmost said clip in said passage out of said passage in a direction angled relative to the longitudinal centerline of said passage.

2. A surgical clip applier, comprising:
a first jaw having a control slot defined therein;
a second jaw having a control slot defined therein; and
a control arm extending into said control slot in each said jaw;
wherein said control arm is movable along said control slots in a first direction to close said jaws and is movable along said control slots in a second direction to open said jaws.

3. The surgical clip applier of claim 1, incorporating by reference all of the elements of that claim; wherein each said clip is independent form and positioned adjacent to at least one other said clip.

4. The surgical clip applier of claim 1, incorporating by reference all of the elements of that claim; further comprising a lower ramp located at the distal end of said passage; wherein frictional force between said distalmost clip and said lower ramp holds said distalmost clip in place relative to said passage.

5. The surgical clip applier of claim 1, incorporating by reference all of the elements of that claim; further comprising an upper ramp generally parallel to said lower ramp and spaced apart from said lower ramp by a distance slightly greater than the thickness of said clips.

6. The surgical clip applier of claim 1, incorporating by reference all of the elements of that claim; wherein at least one said clip is generally U-shaped.

7. The surgical clip applier of claim 1, incorporating by reference all of the elements of that claim; further comprising a clip pusher biased distally, wherein said clip pusher contacts the most-proximal clip in said passage, and wherein force in the distal direction applied by said clip pusher to said most-proximal clip is transmitted through said plurality of clips to said distalmost clip.

8. The surgical clip applier of claim 7, incorporating by reference all of the elements of that claim; further comprising a spring connected to said clip pusher, wherein said spring biases said clip pusher distally.

9. The surgical clip applier of claim 8, incorporating by reference all of the elements of that claim; wherein said pusher is movable into said passage as it urges the distalmost said clip in said passage out of said passage; and wherein motion of said pusher back into said pusher slot allows said clips within said passage to advance distally within said passage.

10. The surgical clip applier of claim 1, incorporating by reference all of the elements of that claim; wherein said pusher slot is underneath and generally parallel to said passage.

11. The surgical clip applier of claim 1, incorporating by reference all of the elements of that claim; further comprising a handle coupled to said pusher, whereby actuation of said handle causes distal motion of said pusher.

12. The surgical clip applier of claim 11, incorporating by reference all of the elements of that claim; wherein said handle includes a source of stored energy, whereby said stored energy is released to urge said pusher distally.

13. The surgical clip applier of claim 2, incorporating by reference all of the elements of that claim; wherein at least one said clip is plastically deformable by said jaws.

14. The surgical clip applier of claim 2, incorporating by reference all of the elements of that claim; wherein the distal end of each said jaw includes a finger angled relative to a remainder of said jaw.

15. The surgical clip applier of claim 14, incorporating by reference all of the elements of that claim; further comprising at least one clip held at an angle relative to the longitudinal centerline of said control arm, and wherein each finger is angled substantially the same as said at least one clip.

16. The surgical clip applier of claim 2, incorporating by reference all of the elements of that claim; further comprising at least one clip held at an angle relative to the longitudinal centerline of said control arm; wherein each finger includes a trench defined therein on the inner surface thereof.

17. The surgical clip applier of claim 16, incorporating by reference all of the elements of that claim; further comprising a pusher slidable to urge the distalmost said clip into position between said fingers along said trenches until said distalmost clip encounters the distal ends of said trenches.

18. The surgical clip applier of claim 2, incorporating by reference all of the elements of that claim; wherein the distal end of each said control slot is located laterally outward from the proximal end of said control slot.

19. The surgical clip applier of claim 2, incorporating by reference all of the elements of that claim; further comprising a handle coupled to said control arm, whereby actuation of said handle causes distal motion of said control arm and closure of said jaws.

20. The surgical clip applier of claim 19, incorporating by reference all of the elements of that claim; wherein said handle includes a source of stored energy, whereby said stored energy is released to urge said control arm distally.
